(19) United States
(12) Patent Application Publication
(10) Pub. No.: US 2004/0091536 A1
(43) Pub. Date: May 13, 2004

(54) Telithromycin suspension with masked taste

(76) Inventors: Julien Meisonnier, Bordeaux (FR); Christophe Lebon, Beneterie (FR); Sandrine Salle, Illiers La Ville (FR)

Correspondence Address: Norman H Stepno, Burns Doane Swecker & Mathis, PO Box 1404, Alexandria, VA, 22313-1404, US

(21) Appl. No.: 10/471,241

(22) PCT Filed: Mar. 8, 2002
(86) PCT No.: PCT/FR02/00835

(30) Foreign Application Priority Data
Mar. 9, 2001 (FR) 01-03234

(51) Int. Cl. A61K 31/7048; A61K 9/24

(52) U.S. Cl. 424/471; 514/27

(57) ABSTRACT

A dry, reconstitutable telitiromycin suspension characterized in that it contains granules or granules comprising a core containing telithromycin which is optionally associated with at least one waxlike compound and optionally with at least one polymer and/or binding agent, and at least three successive layers of coating from the core outwards, said granules being associated with excipients including at least one thickening agent, at least one preservative agent and at least one pH modulator agent.

The present invention relates to a dry, reconstitutable telithromycin suspension with masked taste prepared from granules and coated granulates and capable of oral administration.

Telithromycin, from a family of antibiotics similar to macrolids, ketolids, also blocks protein synthesis and has a wide spectrum of action especially against pathogenic resistant germs of the respiratory tract such as Haemophilus influenzae, Moraxella catarrhalis, and also Chlamydia pneumoniae, Legionella pneumophila and Mycoplasma pneumoniae, and this by per os administration. It is consequently utilised as an antibiotic in the treatment of infections by sensitive germs (EP 680967).

Nevertheless, telithromycin, just like numerous other active ingredients, in particular macrolids, has an unpleasant taste, such that it becomes imperative to mask its taste. Masking of taste is understood to mean any process which effectively retards or prevents the occurrence of an unpleasant taste specific to telithromycin while it is being administered orally.

In the case of formulations administered in the form of liquid, compositions packaged in the form of multidose vials, and in particular in the case of dry suspensions intended for extemporaneous reconstitution, also called reconstitutable dry suspensions, the absence of bitterness must be maintained over a time equivalent either to the duration of the treatment, or to the duration of utilisation of the vial. The granule or coated active granulate utilised in such formulations will thus have to be stable in contact with an aqueous liquid phase over a time at least equal to 24 hours. In practice, this prevents solubilisation of the active ingredient in the liquid phase.

In general, masking of the taste is achieved by encapsulating the active ingredient inside a capsule or by microencapsulation techniques in which a polymer coating is applied to the active ingredient (WO 92/11871).

One of the proposed solutions consists of coating the active ingredient particles by a cellulose polymer. Examples of these polymers especially are ethyl cellulose and hydroxypropyl methyl cellulose.

Another solution consists of coating the active ingredient particle with an acrylic-based polymer. Of these polymers distinguished examples are dependant pH polymers, that is, polymers whose solubility depends on pH and independent pH polymers, that is, insoluble polymers whose intrinsic properties are not influenced by the pH of the medium.

All the same, even if the taste of the active ingredient present in the granules is masked satisfactorily, these polymers interfere with the release of the active ingredient and require the use of agents favouring or retarding solubilisation of the active ingredient and necessitate utilisation of agents favouring or retarding solubilisation of the active ingredient (GB 1 511 852; WO 91/16043).

In addition, conventional techniques and formulae, even though they give good taste masking, do not produce membranes stable in suspension for more than a day.

Matricial microspheres have also been stabilised, but they require a supplementary coating to achieve the desired stability; correct stability can be obtained in pH acid with cellulose acetates, but a release delay is observed (EP 0293885).

Also, there is still considerable need to have a formulation which allows immediate release of telithromycin in a physiological medium, without it having been released in the medium of the formulation, and which exhibits adequate stability, that is, a capacity to conserve masking of the taste over a period at least equal to 24 hours.

Now, the inventors have surprisingly found that a dry, reconstitutable suspension comprising granules or granulates comprising a core containing telithromycin, said core being coated, and excipients appropriate to have a dry, reconstitutable suspension in a liquid medium, allows telithromycin to be isolated for an adequate period to ensure stability of the masking of the taste when the dry, reconstitutable suspension incorporating said granule or coated granulate is reconstituted by adding a defined volume of water when first measured. After absorption, there is immediate release of the telithromycin.

As a consequence, the object of the present invention is a dry, reconstitutable telithromycin suspension characterised in that it contains granules or granulates comprising:

a core containing telithromycin optionally associated with at least one waxlike compound and optionally with at least one polymer and/or with at least one binding agent, and

at least three successive layers of coating from the core outwards,

said granules or granulates being associated with excipients among which there is at least one thickening agent, at least one preservative agent and at least one pH modulator agent.

This association of granules or granulates coated with this type of excipients lends the reconstituted formulation certain organoleptic characteristics, and microbiological stability.

In an advantageous embodiment of the invention, the excipients are in the form of grains associated with granules or granulates.

In another advantageous embodiment of the invention, the successive layers of coating from the core outwards are as follows:

a functional polymer coating (1) optionally containing a waxlike compound allowing immediate release, and

a hydrophobic coating (2) containing at least one waxlike compound, and

a functional polymer coating (3) optionally containing a waxlike compound which can have a structure different to the coating (1), but which also has an immediate release function and conditions the suspension medium.

In terms of the present invention, immediate release is understood to mean release whose kinetics are not substantially modified by the formulation and/or by the parameters of the production process, which indicates that the dissolution profile of the telithromycin depends essentially on its intrinsic properties.

In terms of the present invention, conditioning the suspension medium signifies that the characteristics of the reconstituted suspension, obtained from excipient grains, are selected as a function of the release profile of the granule or active coated granulate, in vitro or after administration of said reconstituted suspension.

In a particular embodiment of the invention, additional layers can be applied whereof the composition is identical to that of the layers (1) and (3).

An overcoating intended to mask possible bitterness associated with the constituents of the third coating layer (3), which does not substantially modify the release properties of the granules and the granulates, can be applied.

In particularly advantageous embodiment of the invention, the core is a preferably spherical neutral substrate of preset granulometry, based on starch, saccharose, ethyl cellulose, lactose or wax, on which telithromycin is applied in layers by pulverisation of a suspension or a solution of said telithromycin, in an aqueous or organic solvent, or in a mixture in the presence of at least one binding agent or at least one polymer or at least one waxlike compound or a mixture of at least two of these agents and optionally lubricants.

In another advantageous embodiment of the invention, the core is telithromycin itself, in the form of crystal, spherical or not, if its granulometry directly produces an efficacious coating. If not an application in layers (montage) of telithromycin will be realised by pulverisation of a solution or a suspension of said telithromycin in the presence of at least one binding agent or at least one polymer or at least one waxlike compound or a mixture of at least two of these agents and optionally of lubricants and organic solvents or water.

In another particularly advantageous embodiment of the invention, the core is a granule based on telithromycin obtained by granulation. The granule can be obtained by humid granulation or in a fluidised air bed or by spherical crystallisation or by emulsion-diffusion of solvent, preferably using (a) solutions of granulations based organic solutions of waxlike compound(s) in the presence of lubricant agents and plasticisers or (b) a polymer such as hydroxypropyl methyl cellulose. In addition, the telithromycin can be assembled by using said granule as a support, by pulverisation of a solution or suspension of the telithromycin in organic solvents or in water, in the presence of at least one binding agent or at least one polymer or at least one waxlike compound or a mixture of at least two of these agents and optionally lubricants.

Apart from telithromycin, the core can contain various agents, including insoluble agents, especially talc, silicone dioxide, titanium dioxide, silica, aluminium, starch and their mixtures; there are also soluble agents, especially mannitol, saccharose, lactose, dextrose, sodium chloride, sorbitol and their mixtures, polyethylene glycol or ampiphilic compounds (magnesium stearate, polysorbates).

The core can contain up to 100% telithromycin, preferably between 60 and 85% as a function of the dosage of the final formulation.

The core containing telithromycin can be of any adapted size, but preferably the size distribution of the core containing telithromycin has an average between 100 to 500 μm, the average preferably being between 100 to 250 μm when the core is a granule or the active ingredient itself, and preferably between 400 and 500 μm when the core is a neutral support on which the active ingredient is applied in layers.

According to the present invention, les coatings (1) and (3) are functional coatings, whereof the aim is to impart an immediate release property of the telithromycin; they are constituted by conventional polymers known to the expert for imparting said properties. Particular examples of polymethacrylates are especially those marketed under the name Eudragit®E and more particularly Eudragit®E 100.

The waxlike agents utilised can be especially selected from the group constituted by waxes, Novata® waxes, gel-waxes and suppowaxes, glyceric macrogol, fatty acids (stearic acid), fatty acid esters, glycerol monostearate Precirol®, Compritol®.

Advantageous examples of these waxlike compounds are hydrophobic waxlike compounds and even more advantageous are hydrophobic waxlike compounds exhibiting low HLB (hydrophilic-lipophilic balance) and having a melting point of between 35 and 53° C., preferably between 37 and 43° C. The waxlike compounds marketed under the names Gelucire® 43/01 and Novata®AB can also be mentioned here, as non-limiting examples.

These waxlike compounds can be associated with glycerol monostearate (GMS).

Accordingly, in a particularly advantageous embodiment of the invention, a coating advantageously made up of a mixture of Eudragit® E100 and optionally hydrophobic waxlike compounds exhibiting low HLB (hydrophilic-lipophilic balance) and having a melting point of between 35 and 53° C., preferably between 37 and 43° C. in the presence of lubricants, can be used as functional coatings (1) and (3). Gelucire® 43/01 and Novata®AB, optionally associated with glycerol monostearate (GMS), can be cited as non-limiting examples.

The rate of coating for the coating (1) (calculated as a percentage (p/p) of dry material applied to the starting substrate) is advantageously between 5 and 100% and preferably between 30 and 60%.

The aim of the hydrophobic coating (2) is to augment the stability of the grain in suspension. It is constituted based on a solution of waxlike compounds in a solvent and optionally comprises a lubricating agent such as for example talc or hydrophobic colloidal silica. The rate of coating for this second coating (calculated as a percentage (p/p) of dry material applied to the starting substrate) is advantageously between 5 and 100% and preferably between 10 and 80%.

Thus, this hydrophobic coating (2) comprises advantageously a waxlike compound or an association of hydrophobic waxlike compounds with low HLB and having a melting point of between 35 and 53° C., preferably 37 and 43° C. in a solvent. Particular examples are Gelucire® 43/01, Gelucire® 53/01, Novata®AB, glycerol monostearate and their mixtures. The functional polymer coating (3) which has release functions complementary to those of the coating (1) is either identical, or similar to said coating (1), but shows the same properties vis-à-vis release of telithromycin and conditions the suspension medium. The rate of coating at this third coating (calculated as percentage (p/p) of dry material applied to the starting substrate) is advantageously between 5 and 200% and preferably between 40 and 100%.

Hypothetically where the coating (3) has a pronounced taste due to the excipients it comprises, an overcoating based on Eudragit® RL30D and RS30D or their mixtures, is applied in the presence of plasticisers and lubricants. The rate of coating at this level will advantageously be between 0 and 15% and preferably between 0 and 5%.

The lubrication agents (lubricants) are advantageously selected from the group comprising talc, hydrophobic colloidal silica and glycerol monostearate.

The plasticisers are advantageously selected from the group made up of dibutyl sebaccate, triethyl citrate, diethyl phthalate, acetyl triethyl citrate, acetyl tributyl citrate, glycerol monostearate (GMS) and Myvacet®.

The granules and coated granulates utilised to prepare the reconstitutable dry suspension according to the present invention are prepared according to a process which comprises making the core or support and optionally includes a complementary assembly stage.

The process can advantageously comprise the following stages:

application of telithromycin solubilised on the support, in the presence of preferably hydrophobic waxlike compounds and/or de polymers, and at least one lubrication agent in a solvent, or a mixture of solvents,

application of a first coating, functional polymer coating (1) and optionally waxlike compounds, said coating enabling immediate release,

application of a second coating, hydrophobic coating (2) containing at least one waxlike compound or an association of waxlike compounds,

application of a third coating, functional polymer coating (3) and optionally waxlike compounds, said coating capable of having a structure different to that of the coating (1), but also exhibiting an immediate release function, and optionally

drying of the resulting granules or granulates.

The coating solvents are those conventionally used by the expert. Examples of these are water, methylene chloride, ethanol, isopropanol and their mixtures.

This process is performed on a fluidised airbed or by any other similar industrial process known to the expert.

The drying operation can be performed on a fluidised airbed, in a rotary dryer under vacuum or using any equivalent technique which aids in removing residual solvents.

When necessary, the process also comprises the application of additional layers identical to layers (1) and (3).

In an advantageous embodiment of the invention the excipient grains contain, apart from a thickening agent, a preservative agent and a pH modulator agent, other excipients selected from among those conventionally used by the expert to make these formulations. Examples of these excipients are sweeteners, fillers, aromatic compositions, colours, surfactants and antioxidants.

This suspension can be obtained in several ways:

by simple addition to the active grain containing telithromycin of excipients in the form of a powder mixture,

by addition to the active grain containing telithromycin of a dry granule of excipients. In this case, the excipients are granules preferably obtained by humid granulation,

by addition to the active grain containing telithromycin of excipients installed on the active grain by a coating process performed advantageously on a fluidised airbed.

By way of thickener, all the thickeners known to the expert can be mentioned, especially those selected from the group comprising gums such as xanthum, guar and tragacanth, magnesium silicate and their associations, sodium alginate, propylene glycol alginate, cellulose compounds such as hydroxyethyl cellulose, hydroxypropyl cellulose, methyl cellulose, carboxymethyl cellulose, carbopols, gelatine, poloxamers, or associations of these compounds and carrageens.

By way of pH adjuster agent, those selected from the group comprising citric acid, soda, sodium citrate, trisodic citrate or any pharmaceutically acceptable compound having the capacity to buffer an aqueous solution can be cited advantageously.

By way of preservative agent examples are those selected from the group comprising potassium or sodium sorbate, sodium benzoate, azorubin, bronopol, ethylene diamine tetraacetic acid (EDTA), methyl, ethyl, propyl and butyl p-hydroxybenzoate (parabens) as well as their salts, used singly or in a mixture, propionic acid, sulphites and cresol.

The suspension nay also contain one or more sweeteners such as saccharin salts and/or potassium acesulfam, or any other sweetener known to the expert such as aspartame, saccharose and its derivatives, trehalose, sodium glycyrrhizinate or their mixtures, an opacifier such as Opadry®OYB or titanium oxides, and capture agents of products such as cyclodextrins whereof the quantities will be adapted as a function of the size of the molecule and of the function to be isolated.

The suspension can also contain one or more aromatic compositions and a filler, in particular polyols, for example sorbitol (Neosorb®), xylitol and lactitol.

The excipient grain can be obtained by a process of humid granulation or any other similar industrial process known to the expert. It can especially be obtained by producing a hydro-alcanolic solution of sweeteners and/or preservatives which serve as wetting liquid for a mixture of filler such as sorbitol, thickening agent, opacifier, pH adjuster agent, optionally aromatic compositions, with the function of the filler to create sufficient mass for granulation. Any other excipient fulfilling the same function could also be utilised.

Another alternative consists of installing the excipients on the active grain by any technique known to the expert, especially on a fluidised airbed.

At the time the medication is first taken, the suspension is prepared by addition of a defined quantity of water (for example in volume, or by a gauge line on the vial), directly to the vial containing the final dry mixture.

The excipient grains prepared in this way allow rapid reconstitution of the suspension, which needs only manual agitation by rotation to homogenise the preparation; in addition the resulting suspension has good bacteriological stability and masking stability greater than 7 days and independent of the pH of the suspension. It is particularly useful in paediatrics and geriatrics.

The pH of the suspension is adjusted to vary between 5.5 and 10, preferably between 8.5 and 10.

Owing to the presence of waxlike agents, the masking stability of the suspensions is improved. The waxlike agents also allow the quantity of polymers used for the coating to be diminished, thus the toxicity induced by said polymers.

The invention and the advantages flowing therefrom will emerge better from the following exemplary embodiments.

### EXAMPLE 1

### Preparation of the Active Grain Without Water

1.1. Preparation of the Active Grain:

Because the telithromycin used has a sufficiently high granulometry, it is installed on the telithromycin alone.

Stage 1: installation

A solution based on telithromycin-Novata® AB-Talc M 10 (57.1%-28.6%-14.3%) in a mixture of methylene-ethanol chloride (50%-50% by weight) is pulverised on the telithromycin grain.

The dry concentration of methylene-ethanol chloride is equal to 43.75% by weight and the dry pulverised/substrate ratio is equal to 131.3% by weight.

Stage 2: coating (1)=functional polymer coating

A solution based on Eudragit® E 100-Novata AB-Talc M 10 (51.4%-5.7%-42.9%) in the methylene chloride is pulverised on the granule obtained in Stage 1.

The dry concentration of methylene chloride is equal to 19.4% by weight and the dry pulverised/substrate ratio is equal to 52.5% by weight.

Stage 3: coating (2)=hydrophobic coating

A solution based on Novata® AB and glycerol monostearate (GMS)-Talc M 10 (51.4%-5.7%-42.9%) in a mixture of methylene chloride-ethanol (70-30) is pulverised on the granule obtained in Stage 2.

The dry concentration of methylene chloride-ethanol is equal to 19.4% by weight and the dry pulverised/substrate ratio is equal to 35% by weight.

Stage 4: coating (3)=functional polymer coating

A solution based on Eudragit® E 100-Novata AB-Talc M 10 (45.7%−11.4%−42.9%) in methylene chloride is pulverised on the granule obtained in Stage 3.

The dry concentration of methylene chloride is equal to 19.4% by weight and the dry pulverised/substrate ratio is equal to 87.5% by weight.

1.2. Preparation of the Grain for Suspension:

The excipients are mixed in quantities giving the following proportions:

| Excipient | Quantity (g) |
|---|---|
| Carrageen | 0.933 |
| Opacifier | 0.933 |
| Sorbitol | 7.6 |
| Aromatic composition | 0.477 |
| Citric acid | 0.013 |
| Sodium nipasept | 0.300 |
| Sodium saccharinate | 0.081 |
| Potassium acesulfame | 0.020 |

1.3. Distribution and Reconstitution of the Suspension:

30.5% of excipient grains and 69.5% of telithromycin grains are introduced to the final packaging (by mixture then simple feeding or double feeding without previous mixing). The vial will be filled as a function of the dose of TLM to be administered and topped up, at the time of use, by mineral water. The reconstituted suspension is stable for at least 7 days.

### EXAMPLE 2

### Preparation of Active Grain in the Presence of Water

This process decreases the granulometry of particles of 200 μm.

Stage 1: this is identical to Stage 1 described in Example 1 above.

Stage 2: Coating (1)=functional polymer coating

A solution based on Eudragit® E 100-Novata AB-Talc M 10 (52.9%-5.9%-41.2%) in a methylene chloride/water (9/1) mixture is pulverised on the granule obtained in Stage 1.

The dry concentration of methylene chloride/water is equal to 18.9% by weight and the dry pulverised/substrate ratio is equal to 43.7% by weight.

Stage 3: Coating (2)=hydrophobic coating

A solution based on Novata® AB and glycerol monostearate (GMS)-Talc M 10 (52.9%-5.9%-41.2%) in a mixture of methylene chloride-ethanol-water (66.5-28.5-5) is pulverised on the granule obtained in Stage 2.

The dry concentration of methylene chloride-ethanol-water is equal to 17.9% by weight and the dry pulverised/substrate ratio is equal to 35% by weight.

Stage 4: Coating (3)=functional polymer coating

A solution based on Eudragit® E 100-Novata AB-Talc M 10 (53.3%-13.3%-33.3%) in a methylene chloride-water mixture (9/1) is pulverised on the granule obtained in Stage 3.

The dry concentration of methylene chloride is equal to 16.7% by weight and the dry pulverised/substrate ratio is equal to 75% by weight.

### EXAMPLE 3

### Solubility and Stability Tests

The stability of the suspension is evaluated in terms of pH, taste masking and dosage of the grained-out telithromycin.

The stability of the granules is evaluated in terms of degradation products, dissolution kinetics, taste and residual solvents.

The suspension according to the present invention is stable for at least 2 months and has durable taste masking.

The dissolution profile measured at pH 1 to 100 rpm is shown in the table below:

Dissolution profile of the suspension
(% of dissolved telithromycin)

| Time (minutes) | After reconstitution | After 5 days |
|---|---|---|
| 5 | 70.0 | 62.8 |
| 10 | 88.8 | 80.2 |
| 30 | 93.9 | 88.2 |
| 60 | 94.0 | 90.3 |

1. A dry reconstitutable telithromycin suspension, characterised in that it contains granules or granulates comprising:
a core containing telithromycin optionally associated with at least one waxlike compound and optionally to at least one polymer and/or to one binding agent, and
at least three successive layers of coating starting out from the core,
said granules or granulates being associated with excipients including at least one thickening agent, at least one preservative agent and at least one pH modulator agent.

2. The dry reconstitutable suspension as claimed in claim 1, characterised in that the excipients are associated in the form of grains.

3. The dry reconstitutable suspension as claimed in any one of claims 1 or 2, characterised in that the successive layers of coating starting out from the core are the following:
a functional polymer coating (1) optionally containing a waxlike compound, enabling immediate release,
a hydrophobic coating (2) containing at least one waxlike compound, and
a functional polymer coating (3) optionally containing a waxlike compound, which can have a different to the coating (1), but which also has an immediate release function and conditions the suspension medium.

4. The dry reconstitutable suspension as claimed in any one of claims 1 to 3, characterised in that the core is a neutral substrate on which telithromycin is applied in layers.

5. The dry reconstitutable suspension as claimed in any one of claims 1 to 3 characterised in that the core is telithromycin itself, in the form of crystal, spherical or not.

6. The dry reconstitutable suspension as claimed in any one of claims 1 to 3, characterised in that the core is a granule based on telithromycin obtained by granulation, by spherical crystallisation or by emulsion-diffusion of solvent.

7. The dry reconstitutable suspension as claimed in any one of claims 1 to 6, characterised in that apart from telithromycin, the core contains various agents.

8. The dry reconstitutable suspension as claimed in any one of claims 1 to 7, characterised in that the core contains up to 100% telithromycin, preferably between 60 and 85% as a function of the dosage of the final formulation.

9. The dry reconstitutable suspension as claimed in claim 1, characterised in that the size distribution of the core containing telithromycin has an average of between 100 to 500 μm.

10. The dry reconstitutable suspension as claimed in any one of claims 5 and 6, characterised in that the size distribution of the core containing telithromycin has an average of between 100 to 250 μm.

11. The dry reconstitutable suspension as claimed in claim 4, characterised in that the size distribution of the core containing telithromycin has an average of between 400 and 500 μm.

12. The dry reconstitutable suspension as claimed in any one of claims 1 to 11, characterised in that the functional coatings (1) and (3) imparting immediate release properties are coatings based on polymethacrylate.

13. The dry reconstitutable suspension as claimed in claim 12., characterised in that the polymethacrylate is Eudragit®E and more particularly Eudragit®E 100.

14. The dry reconstitutable suspension as claimed in any one of claims 1 to 13, characterised in that the waxlike compounds are selected from the group constituted by waxes, Novata® waxes, gel-waxes and suppowaxes, glyceric macrogol, fatty acids (stearic acid), fatty acid esters, glycerol monostearate, Precirol®, Compritol®.

15. The dry reconstitutable suspension as claimed in claim 14, characterised in that the waxlike compounds are waxlike hydrophobic compounds exhibiting low HLB (hydrophilic-lipophilic balance) and having a melting point between 35 and 53° C., preferably between 37 and 43° C.

16. The dry reconstitutable suspension as claimed in claim 15, characterised in that the waxlike compounds are Gelucire 43/01 and Novata®AB.

17. The dry reconstitutable suspension as claimed in any one of claims 14 to 16, characterised in that the waxlike compounds are associated with glycerol monostearate.

18. The dry reconstitutable suspension as claimed in any one of claims 1 to 17, characterised in that the functional coatings (1) and (3) are constituted by a mixture of Eudragit® E100 and waxlike hydrophobic compounds exhibiting low HLB (hydrophilic-lipophilic balance) and having a melting point between 35 and 53° C., preferably between 37 and 43° C. in the presence of lubricants.

19. The dry reconstitutable suspension as claimed in claim 17, characterised in that the waxlike compounds are Gelucire® 43/01 and Novata®AB, optionally associated with glycerol monostearate (GMS).

20. The dry reconstitutable suspension as claimed in any one of claims 1 to 19, characterised in that the rate of coating for the coating (1) is between 5 and 100%, preferably between 30 and 60%, the rate of coating for the coating (2) is between 5 and 100%, and preferably between 20 and 80% and the rate of coating for the coating (3) is between 5 and 200%, preferably between 40 and 100%.

21. The dry reconstitutable suspension as claimed in any one of claims 1 to 20, characterised in that the lubrication agents are selected from the group comprising talc, hydrophobic colloidal silica and glycerol monostearate.

22. The dry reconstitutable suspension as claimed in any one of claims 1 to 21, characterised in that the plasticisers are selected from the group constituted by dibutyl sebaccate, triethyl citrate, diethyl phthalate, acetyl triethyl citrate, acetyl tributyl citrate, glycerol monostearate and Myvacet®.

23. The dry reconstitutable suspension as claimed in any one of claims 1 to 22, characterised in that the excipient grains contain, apart from a thickening agent, one preservative agent and a pH modulator agent, excipients selected from the group comprising sweeteners, fillers, aromatic compositions, colours, surfactants and antioxidants.

24. The dry reconstitutable suspension as claimed in any one of claims 1 to 23, characterised in that the thickener is selected from the group comprising gums such as xanthum, guar and tragacanth, magnesium silicate and their associations, sodium alginate, propylene glycol alginate, cellulose compounds such as hydroxyethyl cellulose, hydroxypropyl cellulose, methyl cellulose, carboxymethyl cellulose, carbopols, gelatine, poloxamers, or associations of these composes and carrageens.

25. The dry reconstitutable suspension as claimed in any one of claims 1 to 24, characterised in that the pH adjuster agent is selected from the group comprising citric acid, soda, sodium citrate, trisodic citrate.

26. The dry reconstitutable suspension as claimed in any one of claims 1 to 25, characterised in that the preservative agent is selected from the group comprising potassium or sodium sorbate, sodium benzoate, azorubin, bronopol, ethylene diamine tetraacetic acid (EDTA), methyl, ethyl, propyl and butyl phydroxybenzoate (parabens), as well as their salts, used singly or in a mixture, propionic acid, sulphates and cresol.

27. The dry reconstitutable suspension as claimed in any one of claims 1 to 26, characterised in that the pH of the suspension is between 5.5 and 10, preferably between 8.5 and 10.
